(19) United States
(12) Patent Application Publication
(10) Pub. No.: US 2026/0041927 A1
(43) Pub. Date: Feb. 12, 2026

(54) PHOTOTHERAPY MASK

(71) Applicant: Chung-Yang CHEN, New Taipei City (TW)

(72) Inventor: Chung-Yang CHEN, New Taipei City (TW)

(21) Appl. No.: 18/796,726

(22) Filed: Aug. 7, 2024

(51) Int. Cl. A61N 5/06

(52) U.S. Cl. CPC A61N 5/0616; A61N 2005/0632; A61N 2005/0647

(57) ABSTRACT

A phototherapy mask includes a transparent mask body, a plurality of strip-shaped light modules, and a headband assembly. The transparent mask body has an outer frame, an outer transparent substrate, an inner transparent partition, and an eye frame. The outer transparent substrate and the inner transparent partition are disposed on a front side and back side of the outer frame, respectively. The eye frame is positioned within the outer frame and connects the outer transparent substrate and the inner transparent partition, forming a mouth-nose opening below the eye frame. The strip-shaped light-emitting modules are disposed on the transparent mask body to project light beams on a face of the head. The headband assembly is attached to the two opposite ends of the outer frame for securing the mask on the head.

### BACKGROUND OF THE INVENTION

### (1) Field of the Invention

The invention relates to a phototherapy mask, in particular to a phototherapy mask with eye openings and a mouth-nose opening.

### (2) Description of the Prior Art

In order to solve the problem of aging appearance, a variety of different treatments have been developed.

Modern people often try various beauty care behaviors to maintain a beautiful appearance. In the field of medical beauty, many studies have pointed out that light beams of different wavelengths will have different stimulating effects on the skin. Therefore, in recent years, it has become popular to use phototherapy to stimulate blood circulation in the skin and to achieve the effect of stimulating collagen proliferation in addition to applying skin care products to maintain the skin.

As mentioned above, since most people's focus of skin care is on their face, various phototherapy masks for irradiating the face with light beams have been introduced in the existing technology. However, existing phototherapy masks are often in the form of opaque masks, and, therefore, users can't observe the condition of their skin in real-time when using them, wherein most of the existing phototherapy masks only have openings at the eyes, and some phototherapy masks do not even have openings at the nose or mouth. Therefore, this kind of phototherapy mask must often be kept at a certain distance from the user's face when wearing it, so that the user can breathe through the gap between the two. In addition, although some phototherapy masks also have a nose opening and a mouth opening, so the user's nose and mouth can be exposed individually when wearing one of them, and, however, since everyone's face is different in size, even if the phototherapy mask has a nose opening and a mouth opening at designated locations, it is easy to encounter the problem that the nose and the mouth cannot be aligned with the nose opening and the mouth opening in actual situation, and it is very inconvenient.

### SUMMARY OF THE INVENTION

In view of the fact that in the prior art, the existing phototherapy masks often have the problem of being opaque, and the nose opening and the mouth opening are also prone to misalignment due to size mismatch. Therefore, the main purpose of the present invention is to provide a phototherapy mask that allows users to observe the actual usage conditions through a transparent soft plate, and the phototherapy can be adapted to users of different face sizes through the design of the mouth and nose openings.

In order to solve the problems of the prior art, the necessary technical means adopted by the present invention is to provide a phototherapy mask, which is used for a user to wear on the user's head. The phototherapy mask includes a transparent mask body, a plurality of strip-shaped light modules, and a headband assembly.

The transparent mask body has an outer frame, an outer transparent substrate, an inner partition, and an eye frame. The outer frame has a front side and a rear side arranged oppositely. The outer transparent substrate is fixed on the front side, and the inner transparent partition is fixed on the rear side. The eye frame is positioned within the outer frame and assembles the outer transparent substrate and the inner transparent partition, the eye frame has an eye opening, and the outer transparent substrate and the inner transparent partition form a mouth-nose opening below the eye frame. The eye-opening and the mouth-nose opening correspond to the eyes, the mouth, and the nose of the head of the user, respectively. A plurality of the strip-shaped light-emitting modules are respectively disposed on the inner side of the outer transparent substrate to project light beams on the face of the head of the user.

The headband assembly is assembled at opposite ends of the outer frame for wearing and fixing on the head.

In one of the subsidiary technical means derived from the above necessary technical means, a plurality of fixing members are set on the inner side of the outer transparent substrate, and the strip-shaped light-emitting modules are respectively fixed on the fixing members.

In one of the subsidiary technical means derived from the above necessary technical means, the headband assembly includes an upper headband assembly and a lower headband assembly. The upper headband assembly is assembled at opposite ends of the outer frame to be fixed above the head. The lower headband assembly is assembled at opposite ends of the outer frame and is located below the upper assembly to be fixed below the head.

Furthermore, the upper headband assembly extends from the transparent mask body along a first direction, and the first direction has an elevation angle. Preferably, the elevation angle is between 5 degrees and 30 degrees.

On the other hand, the lower headband assembly extends from the transparent mask body along a second direction, and the second direction has a depression angle. Preferably, the depression angle is between 5 degrees and 30 degrees.

In one of the subsidiary technical means derived from the above necessary technical means, the mouth-nose opening is diamond-shaped.

The present invention mainly uses a transparent mask body to allow users to observe the actual condition of the face when using it. It can also allow the nose and mouth to breathe through the highly fault-tolerant mouth-nose opening, effectively increasing the convenience of use.

The specific embodiments used in the present invention will be further explained through the following examples and drawings.

### BRIEF DESCRIPTION OF THE DRAWINGS

The present invention will now be specified with reference to its preferred embodiment illustrated in the drawings, in which:

FIG. 1 is a schematic three-dimensional view of the phototherapy mask provided by the preferred embodiment of the present invention;

FIG. 2 is a schematic three-dimensional view showing the strip-shaped light-emitting modules arranged in the transparent mask body in the phototherapy mask provided by the preferred embodiment of the present invention because the outer transparent substrate is a transparent structure;

FIG. 3 is a three-dimensional schematic diagram showing another perspective of the phototherapy mask provided by the preferred embodiment of the present invention;

FIG. 4 is a schematic three-dimensional view showing the strip-shaped light-emitting modules arranged in the transparent mask body in the phototherapy mask provided by the preferred embodiment of the present invention because the inner transparent partition is a transparent structure;

FIG. 5 is a three-dimensional exploded schematic diagram showing the inner transparent partition separated from the outer frame of this embodiment;

FIG. 6 is a schematic diagram of the A-A section of FIG. 2; and

FIG. 7 is a schematic cross-sectional view showing the phototherapy mask provided by the preferred embodiment of the present invention when worn by the user.

### DESCRIPTION OF THE PREFERRED EMBODIMENT

The invention disclosed herein is directed to a phototherapy mask. In the following description, numerous details are set forth in order to provide a thorough understanding of the present invention. It will be appreciated by one skilled in the art that variations of these specific details are possible while still achieving the results of the present invention. In other instances, well-known components are not described in detail in order not to unnecessarily obscure the present invention.

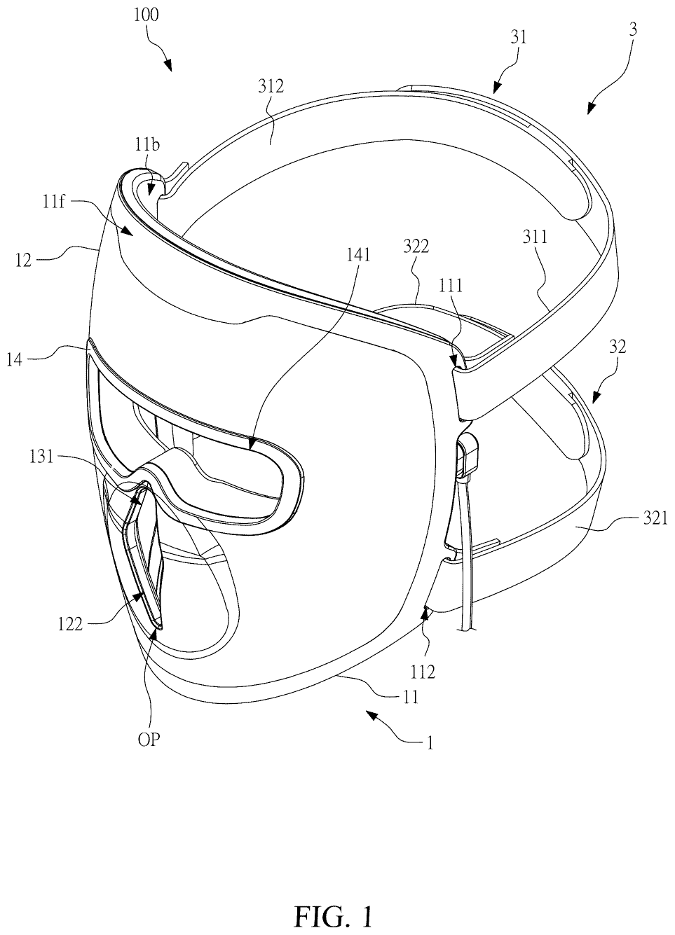

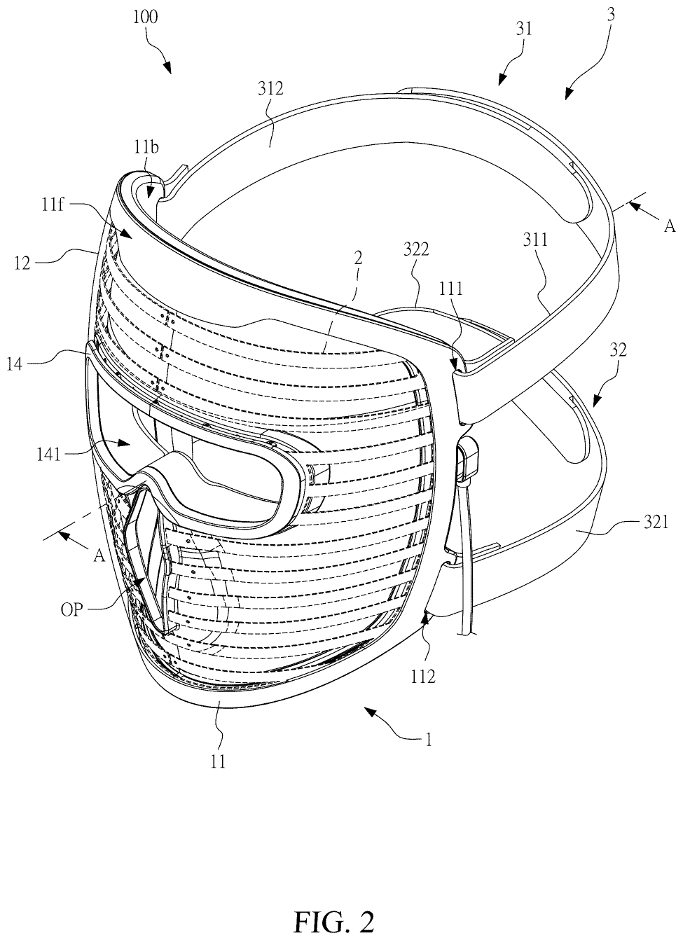

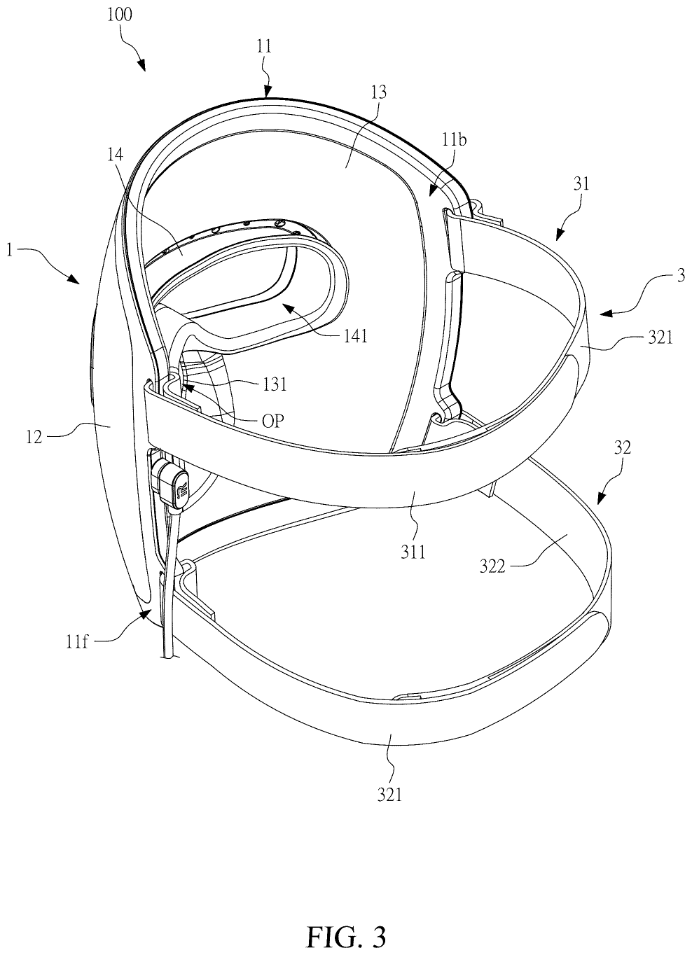

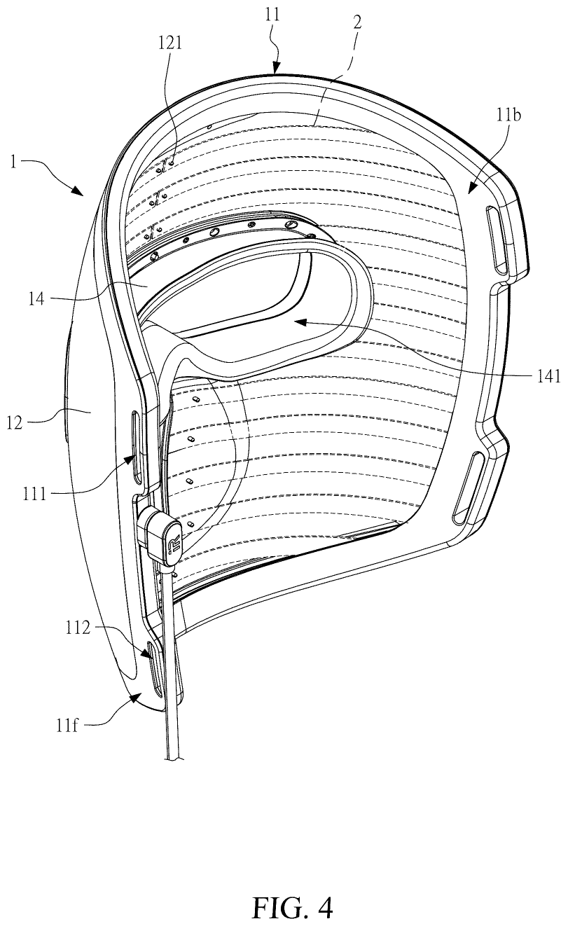

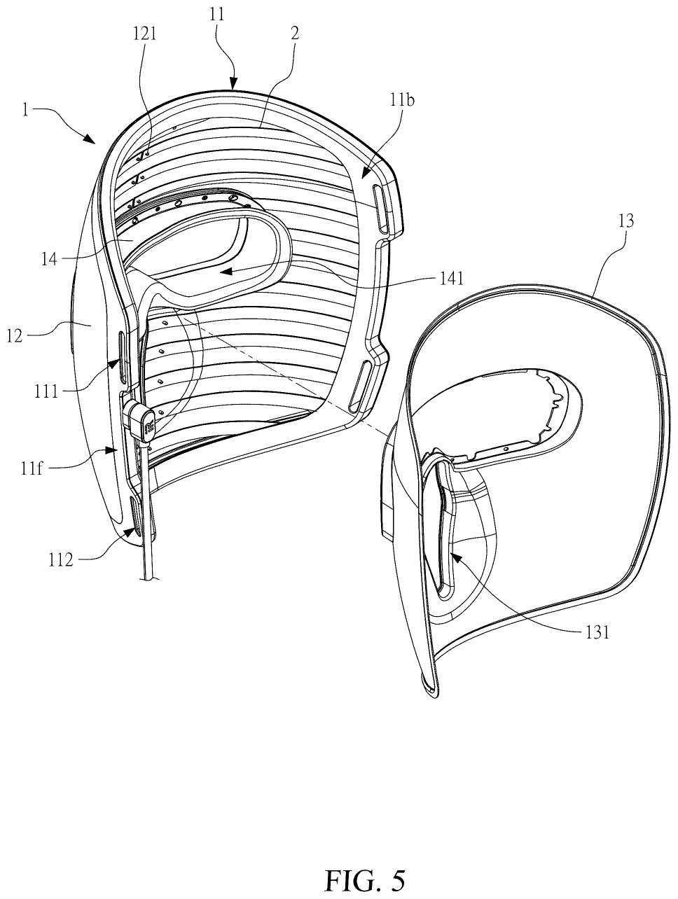

Refer to FIG. 1 through FIG. 5, FIG. 1 is a schematic three-dimensional view of the phototherapy mask provided by the preferred embodiment of the present invention, FIG. 2 is a schematic three-dimensional view showing the strip-shaped light-emitting modules arranged in the transparent mask body in the phototherapy mask provided by the preferred embodiment of the present invention because the outer transparent substrate is a transparent structure, FIG. 3 is a three-dimensional schematic diagram showing another perspective of the phototherapy mask provided by the preferred embodiment of the present invention, FIG. 4 is a schematic three-dimensional view showing the strip-shaped light-emitting modules arranged in the transparent mask body in the phototherapy mask provided by the preferred embodiment of the present invention because the inner transparent partition is a transparent structure, and FIG. 5 is a three-dimensional exploded schematic diagram showing the inner transparent partition separated from the outer frame of this embodiment.

As shown in the FIG. 1 to the FIG. 5, a phototherapy mask 100 includes a transparent mask body 1, a plurality of strip-shaped light-emitting modules 2 (only one labeled in the figures.), and a headband assembly 3.

The transparent mask body 1 has an outer frame 11, an outer transparent substrate 12, an inner transparent partition 13, and an eye frame 14.

The outer frame 11 has a front side 11f and a rear side 11b arranged oppositely. The left and right ends of the outer frame 11 are provided with two upper assembly structures 111 (only one labeled in the figures) and two lower assembly structures 112 (only one labeled in the figures). The two upper assembly structures 111 are relatively disposed above the two opposite ends of the outer frame 11, and the two lower assembly structures 112 are relatively disposed below the opposite ends of the outer frame 11, wherein, in this embodiment, both the upper assembly structure 111 and the lower assembly structure 112 are perforated.

The outer transparent substrate 12 is fixed to the front side 11f of the outer frame 11, and a plurality of fixing members 121 are fixed on the inner side of the outer transparent substrate 12 (only one labeled in the figures). In addition, the outer transparent substrate 12 also has an outer mouth-nose opening 122. The inner transparent partition 13 is fixed to the rear side 11b of the outer frame 11, and is spaced apart from the outer transparent substrate 12. The inner transparent partition 13 also has an inner mouth-nose opening 131.

The eye frame 14 is positioned within the outer frame 11 and assembles the outer transparent substrate 12 and the inner transparent partition 13, and the eye frame 14 has an eye-opening 141. The outer transparent substrate 12 and the inner transparent partition 13 further forms a mouth-nose opening OP connecting the outer transparent substrate 12 and the inner transparent partition 13 by the outer mouth-nose opening 122 and the mouth-nose opening 131 below the eye frame 14.

In this embodiment, the mouth-nose opening OP is diamond-shaped, and through the design of the diamond-shaped opening, the tip of the nose and part of the mouth can be exposed with the smallest and most beautiful geometric design opening, and it makes the transparent mask body 1 closer to the skin of the face, and the phototherapy mask 100 can provide more effective phototherapy power and coverage to the face. In addition, the diamond-shaped mouth-nose opening OP can make the phototherapy mask 100 more comfortable and breathable for the user to wear, and it allows the user to breathe and speak easily while still allowing part of the nose and mouth to remain in the phototherapy mask 100 within the coverage area of phototherapy. In other words, the design of this diamond-shaped opening can bring the phototherapy mask 100 closer to the face, and it makes the light source surface (inner transparent partition 13) closer to the skin of the face, thereby achieving higher and more effective photodynamic efficacy.

The plurality of strip-shaped light-emitting modules 2 are respectively fixed to the fixing members 121, thereby being fixed to the inner side of the outer transparent substrate 12. In this embodiment, the strip-shaped light-emitting module=2 is, for example, an LED light strip.

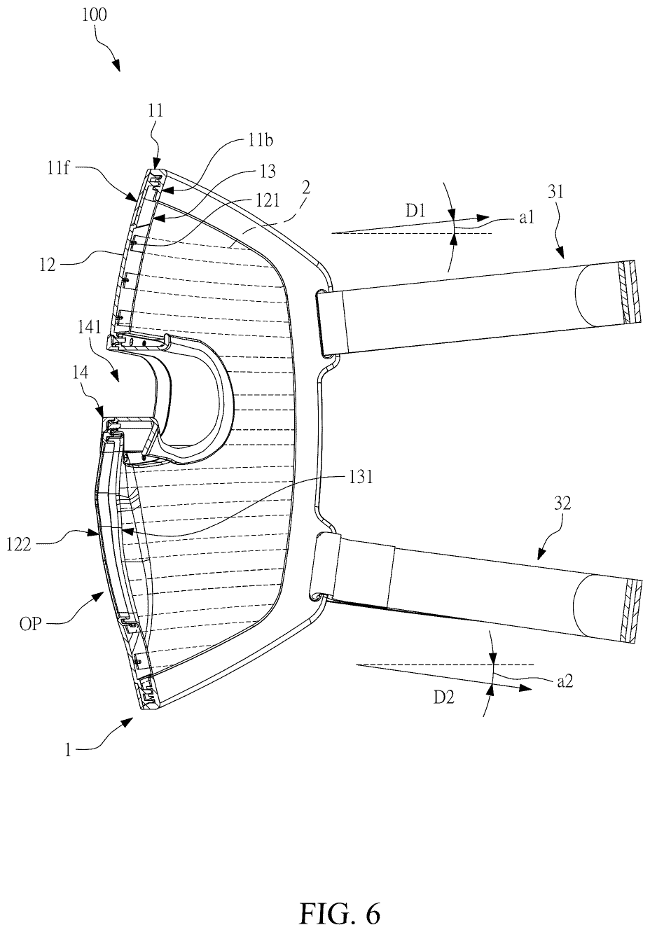

Referring to FIG. 6, FIG. 6 is a schematic cross-sectional view of A-A in FIG. 2. As shown in FIG. 1 to FIG. 6, the headband assembly 3 includes an upper headband assembly 31 and a lower headband assembly 32. The upper headband assembly 31 includes two upper headbands 311 and 312, and the two upper headbands 311 and 312 are respectively assembled to the two upper assembly structures 111. The two upper headbands 311 and 312 respectively extend from the outer frame 11 along a first direction D1, and the first direction D1 has an elevation angle a1. In this embodiment, the elevation angle a1 is, for example, 20 degrees, but it is not limited to this. The elevation angle a1 can also be implemented between 5 and 30 degrees. In addition, the two upper headbands 311 and 312 can be coupled to each other with respect to the other ends of the outer frame 11. In this embodiment, although one end is inserted into the butt hole of the other end to be fixed, it is not limited to this. However, they can be detachably connected through Velcro or fasteners, or even directly connect the two ends with a knot.

The lower headband assembly 32 includes two lower headbands 321 and 322, and the two lower headbands 321 and 322 are respectively assembled to the two lower assembly structures 112 below the upper headband assembly 31. The two lower headbands 321 and 322 respectively extend from the outer frame 11 along a second direction D2, and the second direction D2 has a depression angle a2. In this embodiment, the depression angle a2 is, for example, 20 degrees, but it is not limited thereto. The depression angle a2 can also be implemented between 5 and 30 degrees. In addition, the other ends of the second lower headbands 321 and 322 relative to the outer frame 11 can also be connected similarly to the above-mentioned upper headbands 311 and 312, so no further description will be given here.

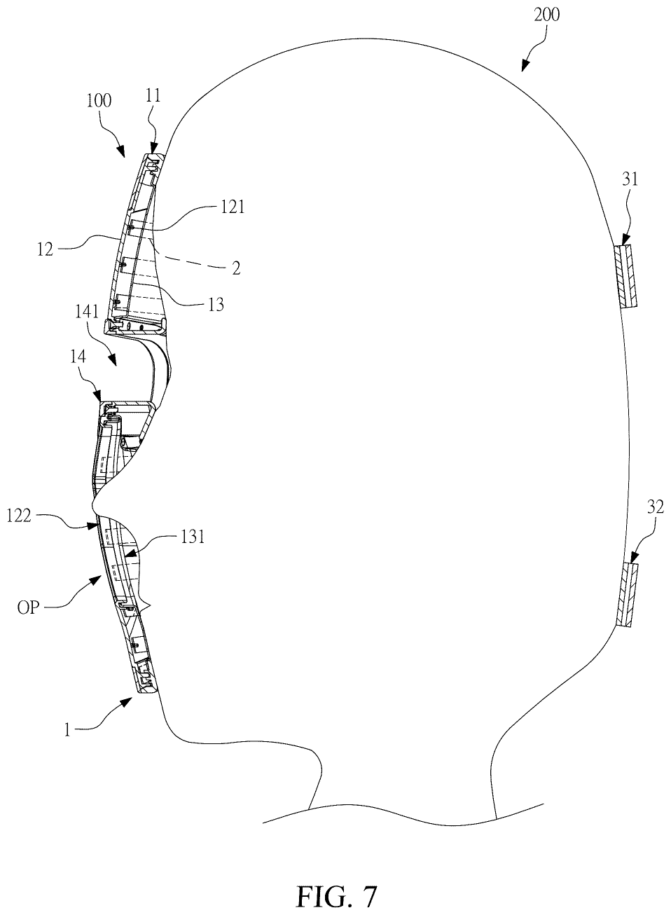

Referring to FIG. 7, FIG. 7 is a schematic cross-sectional view of the phototherapy mask provided by the preferred embodiment of the present invention when worn by a user. As shown in FIG. 1 to FIG. 7, the phototherapy mask 100 of this embodiment is used for a user (not shown) to wear on the user's head 200. When the user wears the phototherapy mask 100 on the head 200 through the upper headband assembly 31 and the lower headband assembly 32, the eye frame 14 will be correspondingly attached to the eyes of the head 200 so that the eyes can see the outside through the eye-opening 141 of the eye frame 14, and when the strip-shaped light-emitting module 2 projects a beam of light toward the face of the head 200, the eye frame 14 blocks the lighting from reaching the eyes.

In addition, when the phototherapy mask 100 is worn on the head 200, the nose and mouth of the head 200 will also be exposed from the mouth-nose opening OP accordingly. Since the mouth and nose opening OP of the phototherapy mask 100 of this embodiment corresponds to covering the nose and mouth of the head 200, even if the size of the phototherapy mask 100 is slightly different from the size of the user's head 200, the fault tolerance rate of the mouth and nose can be improved through the mouth-nose opening OP that spans the range of the nose and mouth so that most users can breathe through the nose or mouth through the mouth-nose opening OP.

In practical applications, the outer frame 11 and the eye frame 14 can have a relatively rigid structure, so that when the phototherapy mask 100 is worn on the head 200, it can withstand the tensile force of the headband assembly 3 without being easily deformed, to avoid the outer frame 11 and the eye frame 14 exert uneven pressure on the face.

As mentioned above, compared with prior art phototherapy masks that have the problem of being opaque and that the nose opening and the mouth opening are often misaligned with the nose and mouth, the light therapy mask of the present invention can indeed allow users to observe the condition of the face through the transparent mask body when the user wears it, to adjust the light irradiation conditions in real-time, and even shut down the light irradiation in an emergency when redness and swelling appear on the face. In addition, since the phototherapy mask of the present invention is also provided with a mouth-nose opening, the nose and the mouth of users with various face sizes can breathe through the highly fault-tolerant mouth-nose opening, and the convenience of use effectively increases.

While the present invention has been particularly shown and described with reference to a preferred embodiment, it will be understood by those skilled in the art that various changes in form and detail may be without departing from the spirit and scope of the present invention.

What is claimed is:

1. A phototherapy mask for a user to wear on the user's head, comprising:
a transparent mask body, including:
a frame, having a front side and a rear side arranged oppositely;
an outer transparent substrate, fixed to the front side;
an inner transparent partition, fixed to the rear side; and
an eye frame, positioned within the outer frame and assembling the outer transparent substrate and the inner transparent partition, and having an eye-opening, and the outer transparent substrate and the inner transparent partition forming a mouth-nose opening below the eye frame;
a plurality of the strip-shaped light-emitting modules, respectively disposed on the inner side of the outer transparent substrate to project light beams on the face of the head of a user; and
a headband assembly, assembled at opposite ends of the outer frame for wearing and fixing on the head of the user.

2. The phototherapy mask of claim 1, wherein a plurality of fixing members are disposed on the inner side of the outer transparent substrate, and the strip-shaped light-emitting modules are respectively fixed on the plurality of fixing members.

3. The phototherapy mask of claim 1, wherein the headband assembly includes:
an upper headband assembly, assembled at opposite ends of the outer frame to be fixed above the head of the user; and
a lower headband assembly, assembled at opposite ends of the outer frame and located below the upper assembly to be fixed below the head of the user.

4. The phototherapy mask of claim 3, wherein the upper headband assembly extends from the transparent mask body along a first direction, and the first direction has an elevation angle.

5. The phototherapy mask of claim 4, wherein the elevation angle is between 5 and 30 degrees.

6. The phototherapy mask of claim 3, wherein the lower headband assembly extends from the transparent mask body along a second direction, and the second direction has a depression angle.

7. The phototherapy mask of claim 6, wherein the depression angle is between 5 and 30 degrees.

8. The phototherapy mask of claim 1, wherein the mouth-nose opening is diamond-shaped.
